(19) United States
(12) Patent Application Publication
(10) Pub. No.: US 2015/0072868 A1
(43) Pub. Date: Mar. 12, 2015

(54) NANOCAPILLARY DEVICE FOR BIOMOLECULE DETECTION, A FLUIDIC NETWORK STRUCTURE AND A METHOD OF MANUFACTURING THEREOF

(71) Applicant: QUNANO AB, Lund (SE)

(72) Inventors: Jonas Ohlsson, Malmo (SE); Mikael Bjork, Lomma (SE)

(73) Assignee: QUNANO AB, Lund (SE)

(21) Appl. No.: 14/394,686

(22) PCT Filed: Apr. 16, 2013
(86) PCT No.: PCT/SE13/50411

§ 371 Date: Oct. 15, 2014

Related U.S. Application Data
(60) Provisional application No. 61/624,533, filed on Apr. 16, 2012.
(60) Provisional application No. 61/660,310, filed on Jun. 15, 2012.
(60) Provisional application No. 61/708,386, filed on Oct. 1, 2012.

(51) Int. Cl. B01L 3/00; C12Q 1/68; G01N 27/447; B01L 7/00

(52) U.S. Cl. CPC B01L 3/502761; B01L 7/525; C12Q 1/686; C12Q 1/6869; G01N 27/44791; B01L 2200/10; B01L 2200/0663; B01L 2200/0652; B01L 2300/0896; B01L 2400/0421; B01L 2300/0838
USPC 506/2; 438/49; 435/6.12; 506/38; 506/9; 435/5; 204/601; 204/451

(57) ABSTRACT

A device includes at least one nanoscale capillary and means for applying an electric voltage, said means being adapted to create an electric field at least in said capillary when said electric voltage is applied, so that, when said electric voltage is applied, a charged molecule or particle placed within the created electric field can be electrically controlled. A fluidic network structure includes the at least one nanoscale capillary. A method of using and manufacturing the fluidic network structure is also described.

### TECHNICAL FIELD

The disclosure relates to a device, a fluidic network structure and a method of manufacturing said structure.

### BACKGROUND

Several methods are known in the art of manipulating position and motion of charged and non-charged objects in the micron- and the high nanometer range. These methods frequently have the purpose of isolating a single, small-sized object so that it subsequently can be studied.

By way of example, optical tweezers may be used to this purpose. Optical tweezers are capable of manipulating dielectric particles by exerting extremely small forces via a highly focused laser beam. Proteins and enzymes are commonly studied by means of these tweezers.

Another technique used is dielectrophoresis whereby a force is exerted on a non-charged, dielectric particle when it is subjected to a non-uniform electric field. Since the strength of the force strongly depends on the medium and particles' electric properties, on the particles' shape and size, as well as on the frequency of the electric field, particles, including nanoparticles, can be manipulated with great selectivity.

Both above-described methods suffer from not being scalable down to low nanometer range, i.e. they are not usable for molecule-sized objects. This inadequacy is owed to the inherent properties of the respective method. In particular, the force required to controllably move, and in a broader sense manipulate, an object is proportional to the volume of the object. Consequently, to be able to employ any of the above techniques in order to, in a controlled fashion, move a molecule having a diameter of 5 nanometer, such as insulin molecule, a certain distance would require 2 million times larger force than to move a 1 micrometer object, such as typical bacterium.

Thus, scientists looking for ways to study individual molecules need to turn to other manipulating techniques for isolating a single molecule of standard size. With this in view, methods are available whereby a single molecule may be immobilised on a dedicated surface or in pores of a gel. However, neither of these methods performs satisfactorily. More specifically, surface immobilisation is rendered unpredictable by amongst other things chemistry of the surface itself, whereas immobilisation by means of a gel is not sufficiently reliable as regards molecule entrapment.

One objective of the present invention is therefore to eliminate at least some of the drawbacks associated with the current art.

Moreover, once an individual molecule has been isolated, it is often required to be able to further manipulate it in a controlled manner, for instance to aggregate it with other molecules of the same kind.

Furthermore, for the sake of efficiency, the process of isolating a desired molecule is preferably to be performed in parallel and result in a large number of individually isolated molecules. Obtaining high degree of parallelization is important not only for the isolation process, but also for the exemplary aggregating process mentioned above.

A further objective of the present invention is to meet these requirements.

### SUMMARY

The above stated objective is achieved by means of an inventive concept comprising a device, a fluidic network structure and a method of manufacturing said fluidic network structure according to the independent claims, and by the embodiments according to the dependent claims. In this context, term fluidic is to be construed as operable by the interaction of streams of fluid. By providing above concept, a reliable, highly scalable solution for control of position and motion of a single charged molecule or particle, such as ion, in a low nanometer range is obtained.

A first aspect of the present invention provides a device comprising at least one nanoscale capillary and means, such as electrodes, for applying an electric voltage, wherein said means are adapted to create an electric field at least in said capillary when said electric voltage is applied. When said electric voltage is applied, a charged molecule or particle placed within the created electric field can be electrically controlled. Here, term electrically controlled charged molecule or particle is to be broadly interpreted as charged entity, molecule, particle, nanoparticle, nanowire or nanostructure whose position and motion are regulated by electricity. For convenience the term electric voltage is used to address the application of an electric field throughout the application, independently of capacitive or ohmic load applications. The terms should be understood to controllably create potential differences in the solution or medium the charged entities are positioned in.

A second aspect of the present invention provides a fluidic network structure comprising at least one nanoscale capillary, wherein said capillary is positioned on a fluidic channel network and said network is positioned on a substrate.

A third aspect of the present invention provides a method of manufacturing a fluidic network structure comprising at least one nanoscale capillary on a fluidic channel network, wherein said method comprises the steps of providing a substrate, growing, subsequently, at least one vertical, essentially one-dimensional nanostructure on said substrate and patterning thereafter a fluidic channel network. The method further comprises the steps of depositing at least one layer of material creating thereby an enclosing integral unit delimited by the material layer and the substrate and, subsequently, removing at least part of the interior of said enclosing integral unit so as to create said capillary and said fluidic channel network.

By applying a suitable voltage using said means, a potential gradient is created. This potential gradient is directed towards the nanoscale capillary and it also extends into the capillary. Thus obtained potential gradient is capable of guiding a single charged molecule passing by, such as for instance negatively charged DNA-molecule, into the capillary, thus causing the entrapment of the molecule. Once in the capillary, the DNA-molecule may be retained therein. More specifically, voltage value in the uppermost section of the capillary is slightly smaller than the voltage value at the bottom of the capillary. In this way, the potential gradient in the capillary is directed from the uppermost section of the capillary towards its bottom. The voltage difference then effectively retains the DNA-molecule within the capillary, i.e. it prevents its exit from the capillary. Same general principle may even be used to displace the retained molecule within the capillary. In the same context, the molecule may be released from the capillary, e.g. by reversing the direction of the potential gradient. An unprecedented degree of control of position and motion of a single charged molecule is hereby obtained. Depending on specific position of the charged molecule it can be made to enter or exit the capillary. In the same fashion it can also be blocked from exiting or entering the capillary.

For even more control of the position and motion of the charged particle such as DNA-molecule, the nanoscale capillary may be integrated into the fluidic network structure that is non-limitatively embodied as an integral unit, i.e. it is made in one piece. When part of the fluidic network structure, nanoscale capillary is positioned on the fluidic channel network that is positioned on the substrate. The interaction between the nanoscale capillary and the network structure may be realized in several ways, e.g. by enabling fluid communication between the capillary and the underlying channel network such that the trapped molecule may, via the channel network, in a highly controlled manner be transported away from the network structure.

Moreover, since the entire fluidic network structure is positioned on the substrate and in all substantial aspects independent of substrate properties, it is possible to transfer the entire structure to another substrate, the properties of which could be tailored for a specific application.

Also, the inventive concept at hand, typically grown on a standard silicon substrate, is compatible with conventional silicon-based semiconductor technologies, why it is readily and at low cost scalable to large diameter wafers.

Further advantages and features of embodiments will become apparent when reading the following detailed description in conjunction with the drawings.

### BRIEF DESCRIPTION OF THE DRAWINGS

FIG. 1 is a perspective view of a nanoscale capillary according to one embodiment of the present invention.

FIG. 2a is a schematical, cross-sectional view of the nanoscale capillary and of means for applying an electric voltage according to one embodiment of the present invention and

FIG. 2b is a schematical, cross-sectional view of a potential gradient created by said means when arranged in accordance with FIG. 2a.

FIGS. 2c-2f are schematical, cross-sectional views illustrating different trap configurations of the nanoscale capillary and of means for applying an electric voltage according to embodiments of the present invention.

FIGS. 3a-3c illustrates a method of manufacturing of said nanoscale capillary according to one embodiment of the present invention.

FIG. 4 highly schematically shows an exemplary portion of the fluidic network structure of the present invention.

FIG. 5 schematically shows an embodiment of a nanosyringe based on a nanocapillary of the present invention.

Different ways to embody the nanosyringe of FIG. 5 are schematically shown in FIGS. 6a-6d.

FIGS. 6e and 6f schematically show single nanosyringe/nanotrap with (6f) and without (6e) integrated electrodes for detection & manipulation of molecules.

FIG. 6g is an organization chart illustrating applications of a nanosyringe/nanotrap/nanocapillary concept.

FIGS. 6h-6j are schematic diagrams of an embodiment of a viral & bacterial detection/diagnostic platform illustrating: (6h) trapping/loading primers specific for certain species via fluidic network, (6i) trapping/mixing in sample DNA into capillaries and (6j) nanoPCR & detection of species in a sample (intercalating dye).

FIG. 6k is a schematic illustration of a method of human Identification: trap target DNA to be analysed, add primers specific to different short tandem repeat (STR) sequences, & run NanoPCR (no gel electrophoresis required).

FIGS. 6l and 6m are schematic illustrations of an embodiment of a of method of single cell drug screening—(6l) a transparent substrate is loaded with e.g., cancer cells that are trapped in microwells matching the syringe layout of the syringe chip (bottom); (6m) the substrate with cells is pressed onto the syringe chip causing the nanosyringes to gently penetrate the cell membrane. Screening can be performed by injecting different drugs/chemicals via the micro fluidic channels (A to E) into the cells. Observation of the cell reaction to the drug can be done through the transparent substrate or from analysis of extracts from the cell using the nanosyringes.

FIG. 6n illustrates an embodiment of a method of Human in vitro fertilization (IVF): use of a nanopipette/syringe to inject male DNA directly into individual egg cells with a controlled amount (only DNA from single sperm), resulting in higher egg fertilization rates and better IVF outcomes than current technology such as ICSI (intracytoplasmic sperm injection.

FIGS. 6o-6q schematically illustrate an embodiment of a method of DNA sequencing sample preparation—(6o) Wrap around electrodes are used to guide a single stranded DNA molecule into the capillary, (6p) Upon successful trapping, the top electrode is configured to block further DNA from entering, and primer molecules are injected via the fluidic network from below the capillary, and (6q) By heating the chip, the primer can hybridize with the captured DNA to form a DNA strand ready for sequencing.

FIG. 6r is a schematic illustration of a nanocapillary lysing and bioassaying device (NLBD) according to an embodiment. The NLBD include an array of 500,000 capillaries and other, smaller arrays.

FIG. 6s is a close up of a 1000 capillary array of the NLBD of FIG. 6r.

FIG. 6t is a photograph illustrating a NLBD mounted on a circuit board.

FIG. 6u is a micrograph illustrating a single capillary.

FIG. 6v is a side schematic cross section of a NLBD.

FIG. 6w is a schematic diagram illustrating an embodiment of a NLBD that includes multiplexing of multiple nanocapillary arrays.

FIG. 6x is schematical diagram illustrating an embodiment of pressure driven flow through a nanoscale capillary.

FIGS. 7A-7D are schematic diagrams illustrating an embodiment of a method of sensing charged particles moving through the wrap around electrode via induced charges on the electrode.

FIG. 8 is a schematic diagram illustrating an embodiment of a nanocapillary device connected to at least one heating element.

FIGS. 9a-9h are schematic diagrams illustrating the control of charged molecules in a nanocapillary device according to embodiments of the invention.

### DETAILED DESCRIPTION

The present invention will now be described more fully hereinafter with reference to the accompanying drawings, in which preferred embodiments are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. In the drawings, like reference signs refer to like elements.

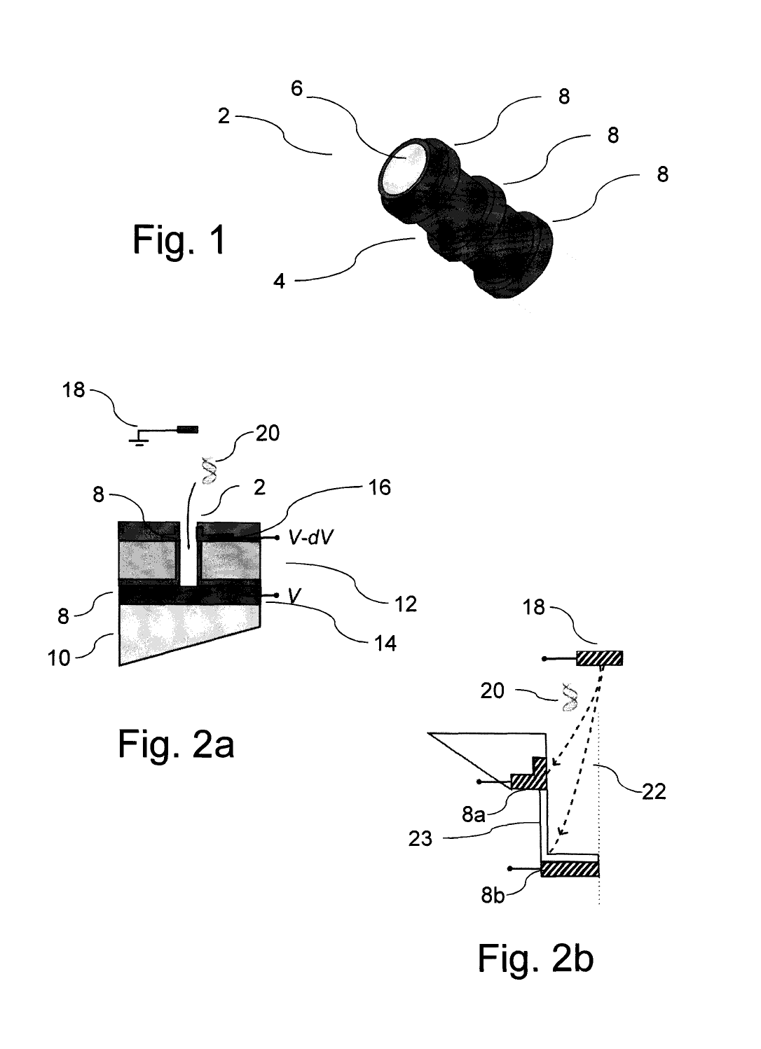

FIG. 1 is a perspective view of a nanoscale capillary 2, i.e. trap or syringe, according to one embodiment of the present invention. Said capillary is delimited by an outer wall 4 of a nanosized tube 6. Said tube has an open upper end and a closed lower end (not shown). Inner diameter of the nanotube is typically 20 nanometer, i.e. the capillary is suitable for accommodation of most species of molecules, but other sizes are conceivable. Its wall is made of an insulator material, typically an oxide, and has a thickness of about 20 nanometer, but other thicknesses may be used. The tube can, depending on application, have a length of a few hundred nanometers up to several microns. In this particular embodiment, means for applying an electric voltage 8 are embodied as three annular structures that are arranged circumferentially on the outer wall of the tube. These annular structures 8 are metallic electrodes, i.e. elements conducting an electric current. These electrodes may be used to induce electric field inside the capillary of the tube. Typically, the nanoscale capillary and the thereto associated electrodes are part of a device (not shown in FIG. 1) used to electrically control position and motion of the charged molecule, such as DNA-molecule.

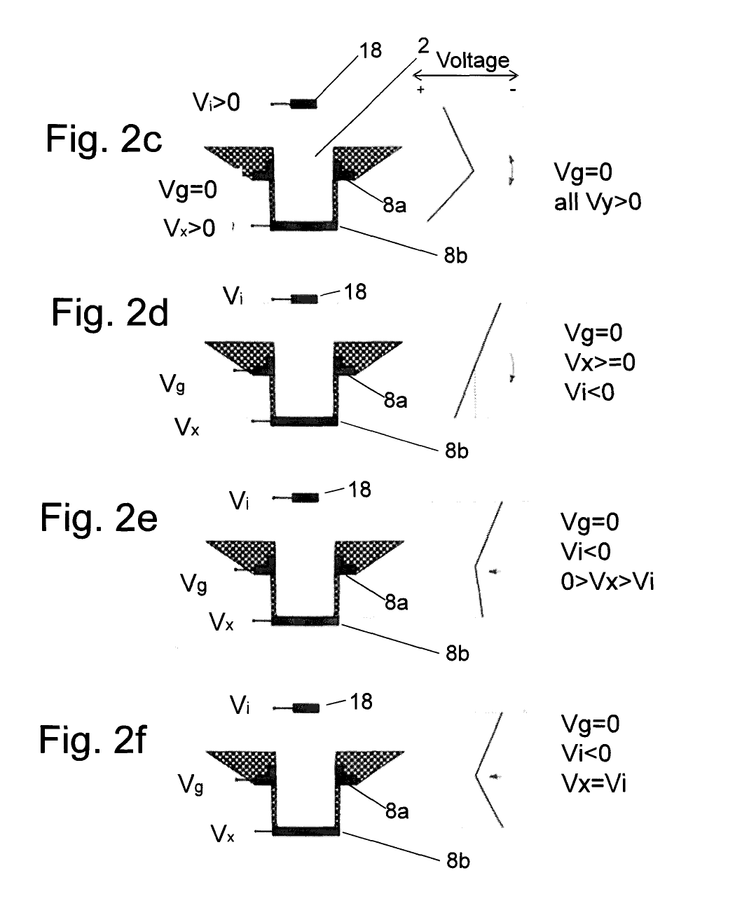

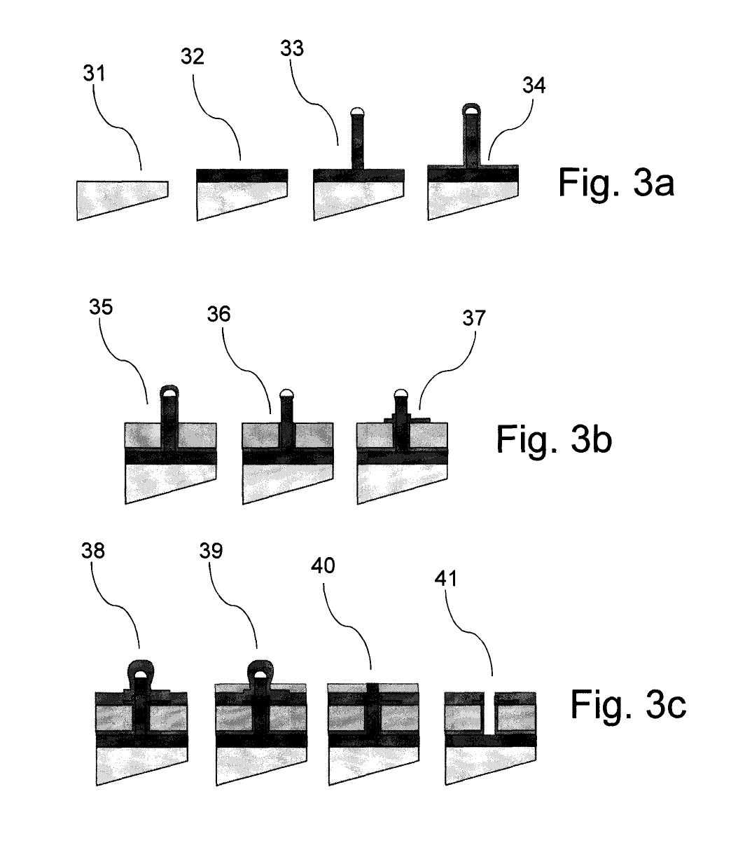

In the following, use of the device 12 comprising the nanoscale capillary according to the above will be described. In this context, FIG. 2a is a schematical, cross-sectional view showing a nanoscale capillary 2 and means for applying an electric voltage 8 according to one embodiment of the present invention. As it can be seen, said device is positioned on a substrate 10, by way of example a conventional Si-substrate. An optional auxiliary layer may be deposited on the substrate. In FIG. 2a the device comprising the nanoscale capillary is positioned on said optional auxiliary layer 14. Here, the auxiliary layer makes up a first electrode and is thus a part of the device, but a solution devoid of an auxiliary layer and having a dedicated electrode positioned adjacent to bottom of the capillary is equally conceivable. A second electrode 16 is positioned close to the open end of the capillary. The first and the second electrodes are active, i.e. they are used to apply a voltage thus inducing electric field inside the capillary of the tube. The applied first and second voltages differ slightly from each other why the electric field (not shown) induced in the capillary has a direction. A third electrode 18, positioned remotely (externally) relative to the capillary is grounded. Consequently, its voltage is zero and it can act as a reference point as regards the induced electric field. In yet another non-limiting embodiment (not shown) the device is provided with only two electrodes 8, 18, one that is grounded 18 and another one 8 that induces the electric field.

Actual trapping of the charged molecule, in this case a DNA-molecule 20 freely diffusing in solution, is illustrated in FIGS. 2b and 2c where means for applying an electric voltage, i.e. the electrodes 8, 18, are arranged in accordance with FIG. 2a. Accordingly, a potential gradient 22 created by the three electrodes 8, 18 can be seen as illustrated with field lines (broken lines) in FIG. 2b. In this embodiment, the two electrodes 8a, 8b of the device are separated by non-conducting sidewall(s) 23 of the nanoscale capillary 2. The non-conducting sidewall(s) 23 and the wrap around electrodes 8a, 8b form a non-conducting/conducting hetero-junction along the longitudinal axis of the capillary 2. This potential gradient is directed towards the capillary 2 and it also extends into the capillary. Thus obtained potential gradient is capable of guiding said diffusing DNA-molecule into the capillary. Since, as is known in the art, a DNA-molecule is negatively charged, a positive voltage (V) is to be applied at the bottom of the capillary and a slightly smaller positive voltage (V-dV) is applied at the uppermost section of the capillary in order to successfully trap the DNA-molecule.

As illustrated in FIG. 2c, a blocking potential can be configured by setting the potential of the external electrode 18 and the upper and lower wrap around electrodes 8a, 8b of the nanocapillary 2. By setting the potential of the external electrode 18 greater than 0 (Vᵢ>0) and the upper wrap around electrode 8a at a lower voltage (such as V_{g}=0), a potential gradient is formed which will block other charged molecules from entering the top of the nanocapillary 2. In FIGS. 2c-2f, the voltage is more positive to the left and more negative to the right. The potential of the lower electrode 8b may also be set to a potential greater (e.g., Vₓ>0) than the potential of the upper wrap electrode 8a. With this configuration of potentials, a charged molecule 20 in the nanocapillary 2 will be blocked from exiting the nanocapillary 2. That is, when V_{g} of the upper wrap around electrode 8a is set at a lower potential (e.g., V_{g}=0) than the potentials (e.g., V_{y}>0, where y is i or x) of the external electrode 18 and the lower wrap around electrode 8b, charged molecules 20 outside of the nanocapillary 20 are blocked from entering the nanocapillary and charged molecules 20 inside the nanocapillary are blocked from exiting the nanocapillary 20.

In the embodiment illustrated in FIG. 2d, the potentials on the external electrode 18 and the wrap around electrodes 8a, 8b are configured for trapping or loading a capillary 2. For example, when Vᵢ<0, V_{g} is greater Vᵢ (e.g. V_{g}=0) and Vₓ is greater than V_{g} (e.g. Vₓ>0), a positively charge molecule 20 will be drawn into the nanocapillary 2.

In the embodiments illustrated in FIGS. 2e and 2f, the potentials on the external electrode 18 and the wrap around electrodes 8a, 8b are configured for displacing one or more molecules within the capillary towards an electrode or trapping or loading and positioning one or more molecules 20 in proximity of a wrap around electrode 8a in the capillary 2. For example, if V_{g}=0, and both Vₓ and Vᵢ are negative, positive charged molecules 20 will be drawn into the nanocapillary 2. The location of the molecule 20 in the nanocapillary can be adjusted by varying the difference in potential between the upper 8a and lower 8b wrap around electrodes.

In an embodiment, the electrode applying an electric voltage 8 is configured to create an electric potential between the capillary 2 and an external location, such that potential field lines originate inside the capillary 2 and end outside the capillary 2. In an embodiment, the device further includes an electrode that applies an internal potential which is situated such that the potential field lines originate and end inside the capillary.

From the above it can be apprehended that the generated trapping force has an electrophoretic component in both horizontal and vertical plane. In this context, electrophoresis is to be construed as motion of particles relative to a fluid under the influence of a spatially uniform electric field. Furthermore, the trapped particle is physically confined to the capillary by means of the wall of the tube delimiting said capillary.

Once in the capillary, the DNA-molecule may be retained therein. More specifically, as long as the direction of the potential gradient doesn't change, the trapped molecule cannot escape from the capillary. By varying the voltage in the longitudinal direction of the capillary, the retained molecule may be displaced within the capillary. Moreover, the trapped molecule may be released from the capillary, e.g. by reversing the direction of the potential gradient.

Although the working of the device has only been described in connection with negatively charged molecule, it is to be understood that the described functionality may be achieved also for the positively charged molecules. Obviously, this would require a polarity change of the applied voltages.

Conclusively, an unprecedented degree of control of position and motion of a single charged molecule is obtained by means of the above-described device. Indeed, charged molecules and particles with a diameter as small as 1 nanometer may be successfully controlled with said device.

In the same context, any of the trapping, retaining, releasing and displacement may be determined by a level of the applied electric voltage or by a frequency of the applied electric voltage. In particular, by suitably adjusting the applied electric voltage, i.e. by matching it with the resonant frequency of the trapped molecule, the trapped molecule may be made to oscillate with large amplitude and, potentially, even exit the capillary. The selectivity of the device may be improved in several ways. Accordingly, the trapping can be tuned, i.e. applied voltages may be so adjusted, that only a predetermined amount of charge is trapped. Moreover, upon capture of a molecule, the applied voltages may be set such that capture of additional molecules is prevented. In this way only a single molecule may be trapped at any instance. The versatility of the device is hereby greatly improved.

The interface between the voltage inducing electrodes and the interior of the capillaries may be tuned from Ohmic behavior to capacitive behavior by adding a non-conducting passivation layer depending on specific applications and/or chemistry. The passivating layer could be deposited using e.g. atomic layer deposition where the exact thickness may be tuned on the atomic level.

In addition, the inventive concept of the present invention is compatible with prevailing CMOS-technology. Accordingly, the substrate may be customized in order to obtain a certain functionality, e.g. control electronics, and be able to control, for instance, voltage-inducing gate electrodes.

FIGS. 3a-3c sequentially illustrate an exemplary, thus non-limiting, method of manufacturing of said nanoscale capillary according to one embodiment of the present invention. For the sake of simplicity, the illustrated method has been, in a non-limitative way, split into three main phases. These are growth of a nanowire, provision of electrodes and creation of a nanoscale capillary itself.

In the first phase, illustrated in FIG. 3a, first of all, a substrate is provided 31. By way of example, said substrate may be made of silicon, silicon-on-oxide (SOI), sapphire or a suitable III-V-compound semiconductor. Obviously, for industrial applications, the substrate may be replaced by a wafer suitable for fabrication of e.g. integrated circuits. In the exemplary method of FIGS. 3a-3c an auxiliary layer is grown 32 on said substrate. Said auxiliary layer acts as a buffer, i.e. it accommodates difference in the crystallographic structure of the substrate and the subsequently grown structures. This layer is typically made in a III-V-compound semiconductor such as InAs. Its thickness ranges from 100 nanometers to one micron. Subsequently, a vertical, essentially one-dimensional nanostructure, such as nanowire or a nanotube, is grown 33 on the auxiliary layer. In this example, said nanostructure is a nanowire grown catalytically in a high-yield VLS-process (Vapor-Liquid-Solid), wherein a gold particle serves as a catalyst and also allows precise positioning of the future nanowire. The diameter of thus grown nanowires is substantially of the same magnitude as the diameter of the catalytic particle. Accordingly, the thickness of the grown nanowire may be precisely controlled. Same is true for its length that is determined by the duration of the growth. For the application at hand, manufacturing of said nanoscale capillary, the required thickness of the nanowire is typically about between 10 and 50 nanometer, whereas length of the grown nanowire lies between 0.5 and 2 microns. Here, the grown nanowire is made of same material as the auxiliary layer (InAs), but any other semiconductor material suitable for nanowire growth is equally conceivable. Once a nanowire of desired dimensions has been grown, a layer of material is deposited 34, typically using Atomic Layer Deposition (ALD), across the auxiliary layer such that the auxiliary layer and the nanowire become completely encapsulated. This material is typically a dielectric, i.e. an electric insulator that can be polarized by an applied electric field. Normally aluminium oxide (Al₂O₃) is used but even other materials having dielectric properties, such as silicon dioxide (SiO₂) and hafnium oxide (HfO₂), may be used. The thickness of the deposited layer varies between 2 and 200 nanometer.

In the second phase, illustrated in FIG. 3b, where the electrodes are provided, another layer is firstly applied 35 on top of the deposited dielectric layer. One purpose of said layer is to provide structural stability. The nanowire thereby becomes at least partially embedded in the applied layer. Said applied layer is in this embodiment made of photo resist material such as S1813 that normally is spun onto the dielectric layer. The previously deposited dielectric is subsequently removed 36 from the non-embedded portion of the nanowire. A further material layer is subsequently deposited 37, at least in the region immediately adjacent to the nanowire. An electrode embodied as a gate electrode that circumferentially surrounds the nanowire is hereby created. By way of example, said gate electrode is made in metal such as tungsten, polysilicon or silicide. In this embodiment, the previously described auxiliary layer makes up a first electrode, but a dedicated electrode grown analogously to the gate electrode and positioned at a distance from said gate electrode, preferably adjacent to the base of the nanowire, i.e. bottom of the future capillary, is equally conceivable.

In the third phase, illustrated in FIG. 3c, where the nanoscale capillary is created, another layer of dielectric such as Al₂O₃ is deposited 38. One of its purposes is to ensure sufficient electric isolation of the previously created gate electrode. Subsequently, another layer of photo resist material is deposited 39. In the next step, the nanowire is rendered radially exposed 40 by removing the uppermost section of the hitherto created structure, for example by using sputtering. In a subsequent step, the nanowire is removed 41, the material of the nanowire is for instance etched (wet or dry) away, thus creating a tube-like nanostructure that substantially delimits the nanoscale capillary. The auxiliary layer acts as an electrode alongside the created gate electrode.

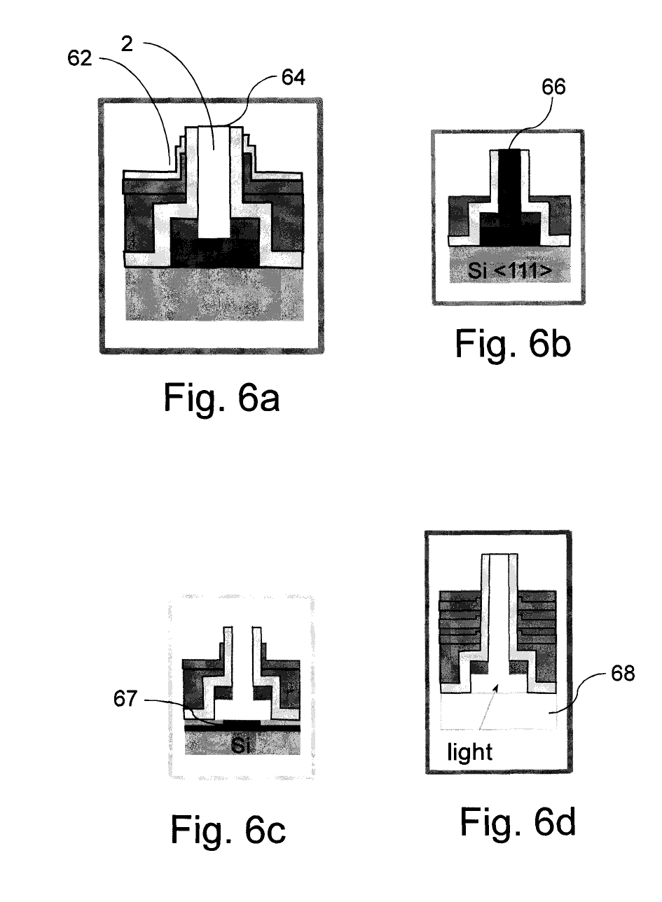

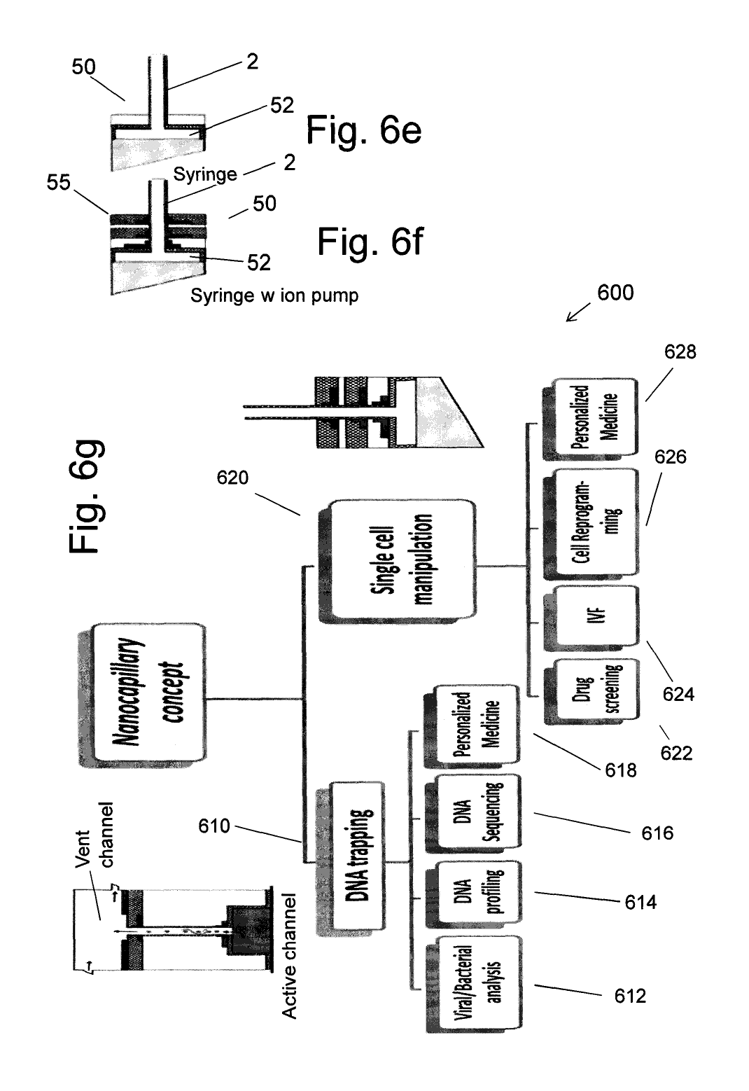

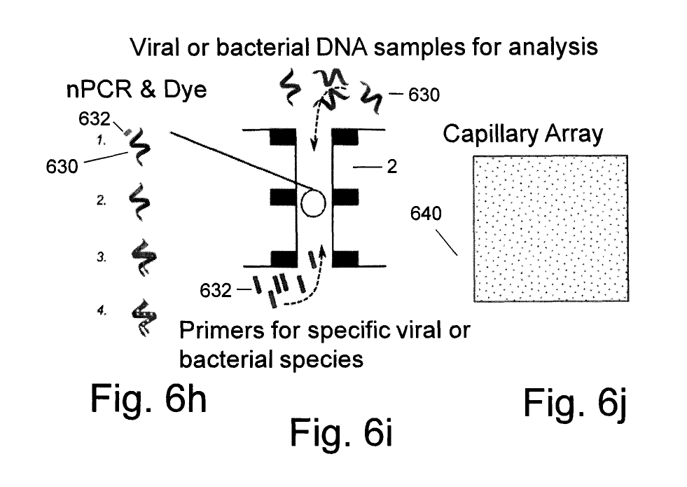

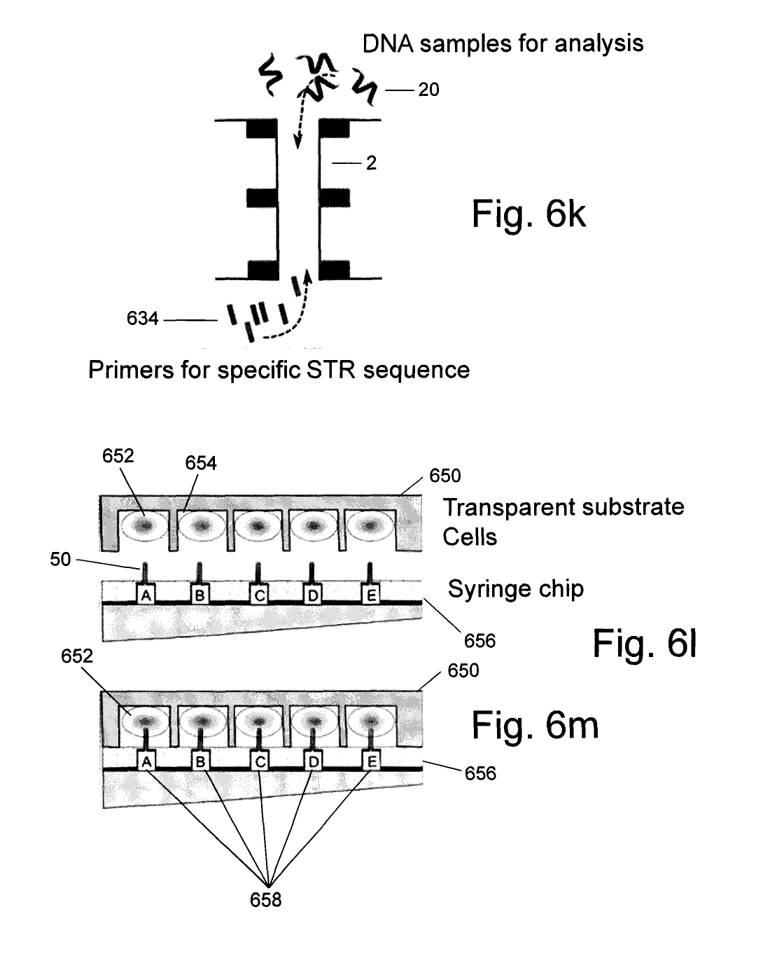

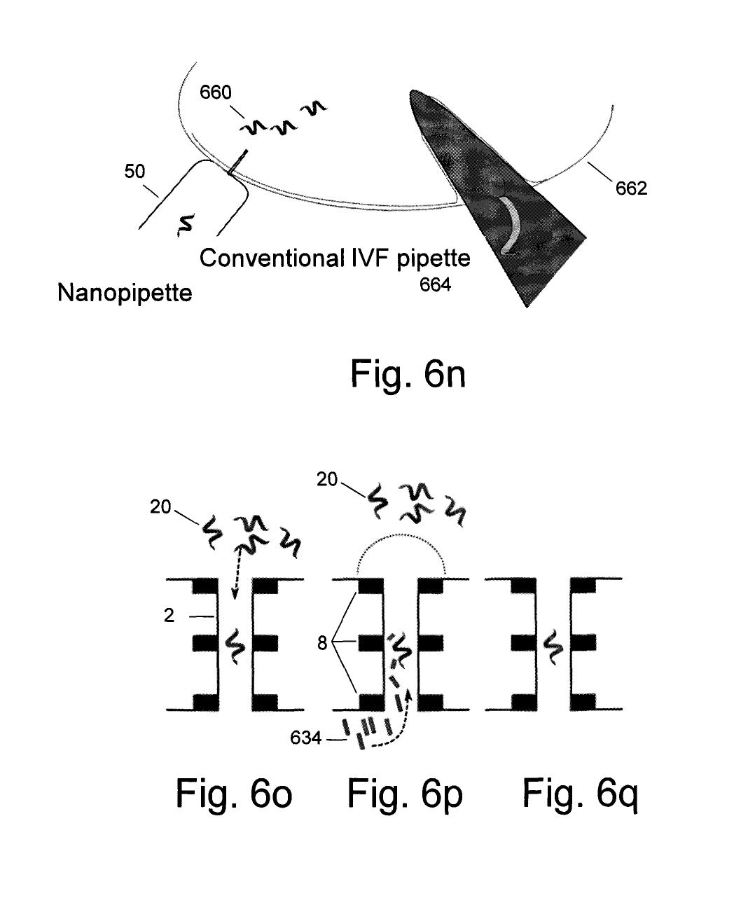

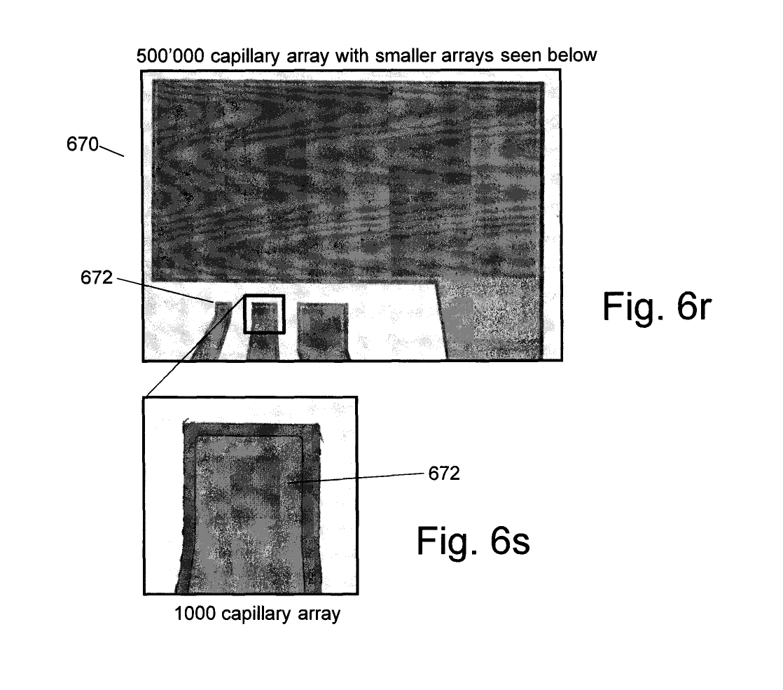

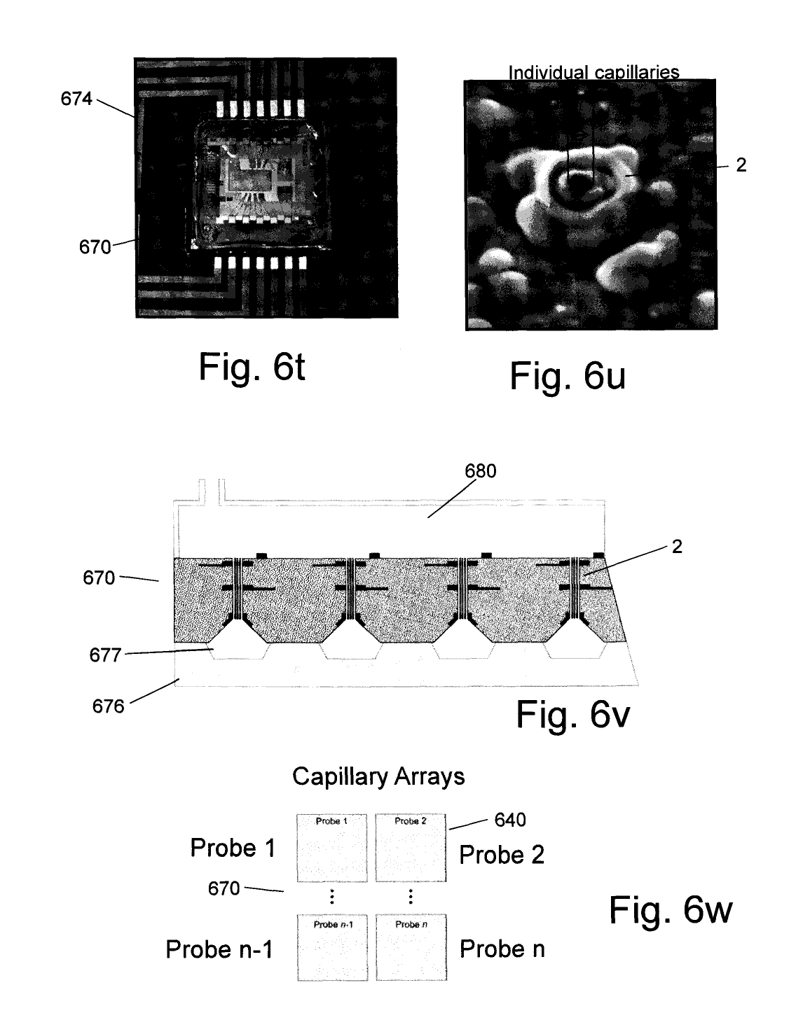

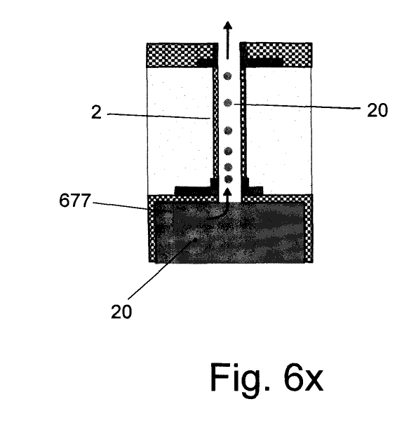

It is to be understood that the method is not limited to manufacturing a nanoscale capillary with a single gate electrode. On the contrary, the above described process of manufacturing of the nanoscale capillary is easily modified so as to include formation of multiple gates. One electrode can be configured to sense charged particles moving through the wrap around electrode via induced charges on said electrode or can be configured as an potential probe such that a change in the concentration of charged particles due to a chemical reaction in the nanocapillary or in the immediate vicinity of the nanocapillary, can be sensed by the sensing electrode or electronic probe (e.g. probe 62 shown in FIG. 6A).

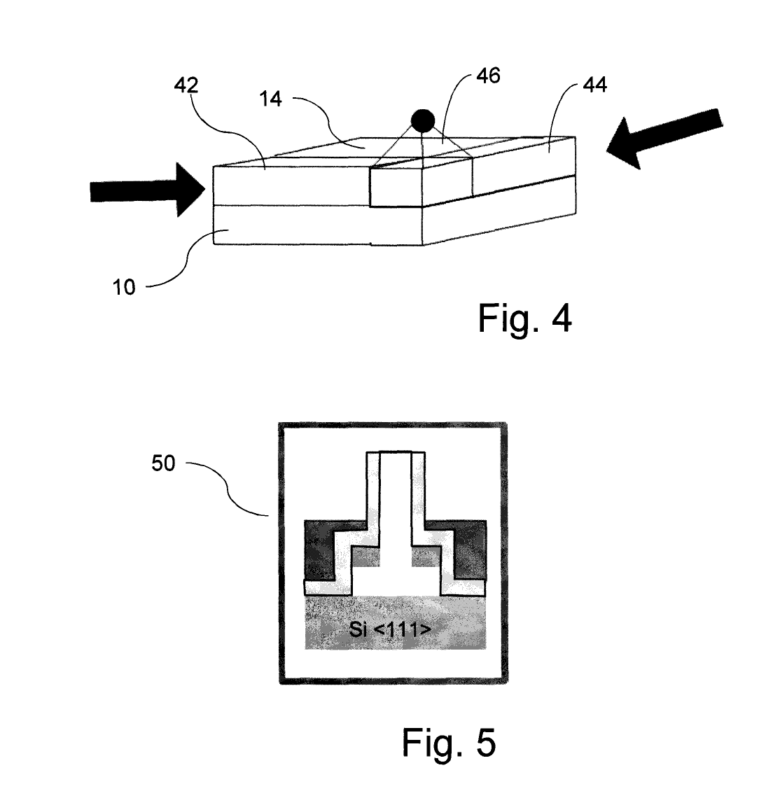

Yet another readily made modification of said process is the creation of an underlying fluidic channel network that is positioned on a substrate or an auxiliary layer. In this way a fluidic network structure comprising the nanoscale capillary delimited by the nanotube and the fluidic channel network is created, wherein said network extends in a substantially horizontal direction and said capillary, as previously discussed, extends in a substantially vertical direction. A highly schematical example of a portion of the fluidic network structure of the present invention is shown in FIG. 4. As it can be seen, two mutually perpendicular channels 42, 44 (indicated by arrows) have been created in the auxiliary layer 14 that is positioned on the substrate 10. In this context, these channels may be so shaped that they vertically extend all the way down to the substrate, i.e. the auxiliary layer is completely removed in this direction. As an alternative, a portion of the auxiliary layer that extends in a vertical direction is preserved and may be provided with functionality, for instance to act as means for applying voltage. The position where the channels intersect is at the same time a position where the removed nanowire had been grown, i.e. the position of the nanoscale capillary. In this embodiment, the outer structure 46 has narrowing shape while its interior (not seen in FIG. 4) is essentially a tube in accordance with previous embodiments. Accordingly, by establishing fluid communication between the capillary and the respective channel, fluids flowing through the respective channel may enter and exit the nanoscale capillary. In this way, charged particles and/or molecules trapped in the capillary in a manner described above in conjunction with FIGS. 2a and 2b may be transported away. This further adds to the versatility of the claimed device. Alternatively, suitable particles and/or molecules can be introduced into the capillary by means of said fluids flowing through the channel structure. This application is of particular interest if these subsurface channels are connected to a reservoir.

The fluidic network structure is achieved by patterning, optionally in the auxiliary layer, a fluidic channel network, depositing subsequently, as described above, at least one layer of material, said material being predominantly composed of a dielectric material, such as Al₂O₃, such that an enclosing integral unit, i.e. unit made in one piece, is created. In next step, at least part of the interior of said enclosing integral unit, i.e. the nanowire as well as at least part of the auxiliary layer is removed via, as previously explained, radially exposed nanowire, e.g. etched away, so as to create said nanoscale capillary and said fluidic channel network.

Above mentioned patterning of the fluidic channel network, comprises, but is not limited to, creating a channel template in the auxiliary layer such that the position where the nanowire has been grown is intersected by at least one section of the channel template. More specifically, channel template is created by providing a resist on at least a portion of the auxiliary layer, forming thereafter a latent image in the resist, e.g. by means of electron beam lithography, and developing subsequently said resist such that appropriate areas of the resist are removed. In a final step the portion of the auxiliary layer corresponding to these removed areas is etched away in the same etching process that removes the nanowire. In this way a fluid communication is established between the capillary and the underlying network of channels enabling hereby streams of fluid to enter and exit the nanoscale capillary. Moreover, for additional control, the substrate and/or the auxiliary layer may be provided with electric and/or fluidic vias. Term via is here to be construed as a substantially vertical connection. Furthermore, electric circuitry may be embedded in the substrate and/or the auxiliary layer. As a result, the trapped charged molecule, such as DNA-molecule, may, via the channel network and the fluidic vias, in a highly controlled manner be transported away from the network structure. This transport is typically controlled by the embedded circuitry. By providing the fluidic network structure comprising the nanoscale capillary for trapping charged molecules and the underlying fluidic channel network, a further means of controlling position and motion of the charged molecule or particle is obtained. The auxiliary layer may also be used for placement of other components such as LED- or HEMT-structures and/or different types of sensors.

In another embodiment, the fluidic network structure comprising at least one nanoscale capillary on a fluidic channel network is manufactured by providing a substrate, growing at least one vertical, essentially one-dimensional nanostructure on said substrate and patterning a fluidic channel network, depositing thereafter at least one layer of material creating thereby an enclosing integral unit delimited by the material layer and the substrate and removing, finally, at least part of the interior of said enclosing integral unit so as to create said capillary and said fluidic channel network.

If called for by a specific application, the fluidic network structure can be custom-made. In this context, if the auxiliary layer is used when growing the fluidic network structure, the custom-made structure may be separated from the underlying substrate. Consequently, thus separated structure is readily transferable from the original substrate to another substrate the properties of which could be tailored, i.e. made any one or a combination of e.g. soft, hard, flexible, opaque, or transparent, in order to make it optimal for the application at hand.

The above described fluidic network structure could with, minor modifications and without departing from the spirit of the invention, become an integrated system with a plethora of fields of application. More specifically, the enclosing structure of such an integrated system should be so shaped that it may function as a nanosized syringe. Such a nanosyringe 50, grown on a Si-substrate, is schematically shown in FIG. 5. By suitably arranging a plurality of these nanosyringes, with or without gate electrodes, on the substrate or auxiliary layer and interconnecting them by means of underlying channel network an integrated system is created. Since nanowire-based nanocapillaries are biocompatible, such a system may find wide use, for example to characterize biology of a cell, for extraction of DNA from cells and for drug injection into single cells. More specifically, by connecting channels of said system to a reservoir and by non-invasively penetrating cells by means of nanocapillaries, it becomes possible to inject molecules into cells themselves. By way of example, molecules freely diffusing in solution stored in the reservoir could be driven into the channel network and subsequently into nanocapillaries by means of electrophoresis or pressure driven flow. Said system may also find applications in microfluidics and genome sequencing. The system of this kind provides a convenient platform for handling liquids, gases, a mixture of both, as well as liquid or gaseous suspensions and aerosols.

For high-capacity applications, the integrated system is inherently capable of considerable throughput. More specifically, by creating entire arrays of nanocapillaries at predetermined positions as well as a grid-like channel network and allowing, for each nanocapillary, that two sections of the channel network intersect at this predetermined position, thus effectively connecting all nanocapillaries, massive parallelisation is achieved. In this way, a huge number of specimens may be analysed and/or managed simultaneously. This parallelisation may, as discussed above in conjunction with FIG. 2, be complemented by providing said system with various types of selectivity, for instance selectivity as regards amount of charge and/or size of particles or molecules, thus further improving its performance.

The integrated system may comprise combination of at least one capillary array connected to at least one of the following parts: a chamber, a reservoir a fluidic channel, a fluidic network, a heater, a temperature sensor, a control chip, or a ccd chip. It is also feasible to make the system modular, where one or more of the above parts can be detached or replaced by a different part. In this way, the capillary array can be configured with different parts for different functionality.

Charged test molecules or particles can be, but are not limited to, DNA, RNA, protein, bacteria, fungi, functional molecules, buffers, enzymes, chemicals, labels, primers. Some of these may be distributed through hydrostatic pressure/flow.

Transport functions include load, hold, release, inject, enter, exit, block, select and isolate. Some functions, as “transfer” and “inject” can be made through hydrostatic pressure as well as electrically.

Functional reactions, functionalizations or manipulation and analysis can be performed in capillaries or in chambers, or fluidic channels, including but not limited to, PCR, qPCR, marking, hybridization, melt analysis, transcription and reverse transcription.

As shown above, positioning of the nanowires, and consequently positioning of the nanocapillaries, but also extension of the channel network in such a system may be deterministically controlled. This is particularly useful for applications demanding high accuracy. By way of example, these requirements are pertinent in situations where the entire integrated system is to be located on a wafer, a so called system-on-a-chip solution. The system of this kind would then typically include even control electronics.

Moreover, another conceivable application, shown in FIG. 6a, is to use the created nanosyringe and make an electronic probe 62. This is achieved by depositing a metallic layer 64 on top of the nanocapillary. Optionally, and schematically shown in FIG. 6b, interior of the nanocapillaries may then be filled with a conductive material 66, either metal or degenerately doped semiconductor. Syringe based on such a nanocapillary could also be fitted with any number of gates. The electronic probe 62 may be used to sense charged matter being injected or extracted into/out of cells. The electronic probe 62 may be a wrap around electrode around the capillary.

A nanosyringe positioned on an auxiliary layer, said layer being used for placement of components such as LED- or HEMT-structures 67 and/or different types of sensors is schematically shown in FIG. 6c.

For applications within the field of optics, a substrate in transparent material 68 may be chosen. Moreover, interior of the syringe may be filled with a transparent material. This configuration, comprising a plurality of gate electrodes, is schematically shown in FIG. 6d.

Additional embodiments are illustrated in FIGS. 6e-6x. Some of the salient characteristics of these structures include:
Nanosyringes and nanocapillary traps can be produced via a combination of bottom-up, top down process in very large arrays (more than 1 billion/cm²) that will allow for massively parallel (or massive sequential) processing;
Individual nanosyringes and nanocapillary traps can be synthesized with selectable internal diameter cavities of as small as 10 nm but with lengths as long as several microns;
Multiple wrap-gate- or ring-electrodes can be integrated with each nanosyringe 50/nanotrap to provide control flows of molecules, proteins, viruses and DNA strands via ion pumps 55 (see FIGS. 6e and 6f). In the embodiments illustrated in FIGS. 6e and 6f, the nanocapillary 2 is seamlessly connected to at least one nanoscale chamber or well 52. Alternatively, the nanocapillary 2 and/or the nanoscale chamber 52 is seamlessly connected to a nano-channel network. As used herein, the term “seamless” means that an attachment boundary or adhesive layer (i.e., the seam) is not present between the nanocapillary 2 and the chamber or network. The seamless connection of the nanocapillary 2 and the nanoscale chamber 52 may be fabricated, for example, by removing at least a portion of the auxiliary layer (formed in FIG. 3a, step 32) below the nanocapillary 2 when removing the nanowire to create the nanocapillary (FIG. 3c, step 41). In this manner, the nanocapillary 2 and the nanoscale chamber 52 are made in the same step, preventing the formation of a seam that would be created if the structure had been formed by bonding a separate substrate with a nanoscale chamber 52 to a nanocapillary device removed from a growth substrate.
Nanofluidics channels and networks can be fabricated between and among the syringes/traps, if desired, to deliver or remove different possible drug combinations, cells or other matter;
Individual nano-device control is possible;
Synthesis is achieved on low-cost and readily available large diameter silicon substrates using a common Metal-Organic Chemical Vapor Deposition (MOCVD) reactor chambers, allowing for development of very capable “labs-on-a-chip.”

The nanoscale chamber 52 of the devices illustrated in FIGS. 6e and 6f may be fabricated as follows. The deposition of the non-conducting material (see, FIG. 3a, step 34) surrounding the nanowire illustrated in FIG. 3a, step 33 forms the walls of the nanocapillary and optionally the top wall of the chamber 52. The nanowire and at least a portion of the buffer layer are then etched in step 41 illustrated in FIG. 3c. The chamber 52 is formed in the location where the buffer layer has been etched away. In this way, the capillary 2 and the walls of the nanoscale chamber 52 and/or fluidic network are formed in a single deposition and etching process. That is, this process does not require two or more parts fabricated separately and then bonded together. However, in an alternative embodiment, the nanoscale chamber(s) 52 may be fabricated in a separate substrate from the growth substrate of the nanocapillaries. In this method, the nanocapillaries are separated from the growth substrate and then bonded to the separate substrate containing the nanoscale chambers 52.

The high aspect ratio and the nm-scale diameter of the capillaries are well suited for manipulation and detection of molecular strands such as DNA and protein. The present inventors have successfully demonstrated the ability to capture DNA strands within the capillaries and DNA strand detection through measurement of electrical charge. By using high density capillary arrays (millions-billions per cm²), with each capillary being addressable, this can be configured as a molecular bio-processor with both parallel and sequential capability.

FIG. 6g is an organization chart 600 illustrating applications of a nanosyringe/nanotrap/nanocapillary concept. Embodiments include DNA trapping 610 and single cell manipulation 620. Embodiments of DNA trapping 610 include field analysis of DNA from viruses or bacteria 612 to facilitate and speed diagnostics of infectious disease, instant DNA profiling 614 eliminating electrophoresis, DNA filtration and preparation for DNA sequencing 616 and personalized medicine 618, just to mention a few. Nanosyringes may be used to controllably inject and extract molecules into and out of living cells without cell rupture and damage. Embodiments of single cell manipulation 620 include drug screening 622, in vitro fertilization (IVF) 624, cell reprogramming 616 and personalized medicine 628.

The bio processor chip can be configured such that no specimen is lost, e.g., within a bio sample—all DNA molecules can be detected and processed if desired. This paves the way for breakthroughs in areas where sample specimen is very limited, including hard to reach tissue biopsy cells such as brain cancer. It could also create breakthroughs in applications in which it is desired to “catch all” bio matter and process it. Applications here may include forensic crime scene investigation, bioterrorism detection, and detection of explosives. Other significant short term applications include filtration and sorting of proteins.

The bio chip can be integrated with more advanced micro fluidic networks on the same chip to be used in personalized drug and medicine applications delivered at point-of-care.

The following are exemplary applications:
Trapping and field (point-of-care) lab-on-chip analysis of DNA from virus or bacteria to assist and speed diagnostics of infectious deceases e.g., HIV and other viral conditions;
On-the-spot (point-of-care), inexpensive, quick turnaround paternity identification (lab-on-a-chip);
Forensic field DNA identification for police and other law enforcement and incarceration agencies (lab-on-a-chip). Like the fingerprints that came into use by detectives and police labs during the 1930s, each person has a unique DNA fingerprint. Unlike a conventional fingerprint that occurs only on the fingertips and can be altered by surgery, a DNA fingerprint is the same for every cell, tissue, and organ of a person. It cannot be altered by any known treatment. Consequently, DNA fingerprinting is rapidly becoming the primary method for identifying and distinguishing among individual human beings.” DNA Fingerprinting in Human Health and Society at http:// www.accessexcellence.org/RC/AB/BA/DNA_Fingerprinting_Basics.php See also DNA Forensics at www.ornl.gov/sci/techresources/Human_Genome/elsi/forensics.shtml.
Preparation of samples for DNA sequencing
Injection of specimens into cells for massively parallel or sequential single-cell studies for drug development and/or drug screening
Improved assisted human in vitro fertilization process (insertion of DNA from a single sperm into a single egg)
Personalized medicine diagnostics to amplify tissue results for hard-to-reach cells such as brain cells, etc.
Other diagnostics and site-specific therapeutics
Full DNA sequencing, significantly faster and much less expensively than possible today

These applications are illustrated in FIGS. 6h-6q. For example, as illustrated in FIGS. 6h and 6i, viral or bacterial DNA 630 may be provided to a nanoscale capillary 2 according to an embodiment. Next primers 632 for the detection of specific viral or bacterial species are added to the nanocapillary 2. A polymerase chain reaction (illustrated in FIG. 6h) may be performed in the nanocapillary 2 to amplify the DNA 630 to aid detection. Optionally, a dye molecule, such as a fluorescent molecule, may be attached to the primer to further aid in detection of the DNA 630. In an embodiment illustrated in FIG. 6j, an array 640 of nanocapillaries 2 is provided on a substrate. In this embodiment, different primers 632 may be provided to each nanocapillary. In this manner, a sample may be analyzed for several different viruses or bacteria at the same time. FIG. 6k illustrates an embodiment of a method of human identification. Target DNA 20 is first trapped in the nanocapillary 2. Primers 634 specific to different short tandem repeat (STR) sequences are then added and NanoPCR is run. In this method, no gel electrophoresis is required.

FIGS. 6l and 6m illustrate an embodiment of a method of single cell drug screening. In the method of FIG. 6l, a transparent substrate 650 is loaded with e.g., cancer cells 652 that are trapped in microwells 654 matching the syringe layout of the syringe chip 656. As illustrated in FIG. 6m, the substrate 650 with cells 652 is pressed onto the syringe chip 656 causing the nanosyringes 50 to gently penetrate respective the cell membrane of a respective cell 652. Screening can be performed by injecting different drugs/chemicals via the micro fluidic channels (A to E) into the cells. Observation of the cell reaction to the drug can be done through the transparent substrate or from analysis of extracts from the cell using the nanosyringes 50. The device illustrated in FIGS. 6l and 6m is a fluidic system which includes an array of nanocapillaries and at least one adjacent structure including any of a chamber, a sample plate, a biocell holder, a channel network, temperature sensor, heating element, cooling element or an optical detector.

FIG. 6n illustrates an embodiment of a method of human in vitro fertilization (IVF). In this method, a nanopipette/syringe 50 is used to inject male DNA 660 directly into individual egg cells 662 with a controlled amount (e.g., only DNA from single sperm). The result is higher egg fertilization rates and better IVF outcomes than current technology such as intracytoplasmic sperm injection (ICSI) 664.

FIGS. 6o-6q schematically illustrate an embodiment of a method of DNA sequencing. As illustrated in FIG. 6o, the DNA 20 to be analyzed is provided to a nanocapillary 2. Wrap around electrodes 8a, 8b are then used to guide a single stranded DNA molecule 20 into the capillary 2. In this embodiment, the DNA is charged. A potential is setup between the wrap around electrodes 8a, 8b which attracts the charged DNA molecule 20. Upon successful trapping, the top electrode 8 is configured, e.g. the potential between the top electrode 8 and the upper electrode 8b is reversed, to block further DNA from entering, and primer molecules 634 are injected via the fluidic network from below the nanocapillary 2 in FIG. 6p. If a DNA molecule is inside the capillary 2, when the potential is reversed, it is blocked from leaving the nanocapillary 2. By heating the chip, the primer 634 can hybridize with the captured DNA 20 to form a DNA strand ready for sequencing in FIG. 6q. The DNA molecule 20 may then be caused to exit the nanocapillary 2 by adjusting the voltages of the top and lower electrodes to produce an electrical gradient which induces the charged DNA molecule to exit the nanocapillary 2.

Additional embodiments are illustrated in FIGS. 6r-6w. These embodiments relate a NLBD, a molecular analyser device combining the multiplexing capability of a bioassay with the sensitivity and quantification ability of q-PCR. The NLBD is a hand-held, all-in-one unit which can be connected to a computing device through a USB connection or Bluetooth wireless connection. Embodiments of the NLBD include the ability to lyse cells, perform PCR and bioassaying. The q-PCR is performed electronically within the chip and the sensitivity of the nanocapillary-sensors makes it possible to obtain results (positive/negative) within 5 minutes or less. The cells may be lysed in a reaction chamber 680 (FIG. 6v) located the hand held device or located on the same chip as the nanocapillary arrays. In an embodiment, lysing may be performed in the nanocapillaries (e.g. a lysing agent is added to the nanocapillaries in addition to the biomaterial to be lysed), such as with small biological entities, such as viruses. The core technology is based on a nanocapillary-sensor array synthesized from sacrificial nanowires that can attract, trap and sense molecular matter electronically in massive arrays of as many as 2 billion devices per cm². The unit may have different configurations, dependent on the users' need/desire for multianalyte DNA detection.

Applications of the NLBD include, but are not limited to:
Multianalyte DNA detection, Point-of-Care;
Fast, high sensitivity multianalyte amplified DNA detection on miniature biochip
Bio chip features
   1. Sample chamber for DNA analytes
   2. Fluidic chamber/network for PCR chemistry
   3. DNA nanocapillary trap and electrical detection system
qPCR capability with electrical read out
Massive multiplexing capabilities with isolated nanocapillary arrays
Combines the multiplexing strength of assays with the sensitivity and quantification ability of PCR in a confined, miniature biochip
All bio-matter is confined in one unit, and the analysis is automated and controlled via USB connection or Bluetooth wireless connection
Results (negative/positive) in 5 minutes or less

Advantages of the embodiments illustrated in FIGS. 6r-6w include:
Handheld, Portable Unit
Automated and computerized
Rapid analysis (5 min)
Confinement of biomatter
No contamination
No loss of amplified DNA
Specificity 100%—No false positives
High sensitivity 99%—No false negatives
Minimal chemical usage (pico-liter)
Low cost (no expensive optics)

FIG. 6r is a schematic illustration of a nanocapillary lysing and bioassaying device 670 (NLBD) according to an embodiment. In an embodiment, the NLBD 670 may include an array of thousands (e.g. 50,000 to 5 million, such as 100,000 to 1 million, for example 500,000) capillaries and may optionally include other, smaller arrays 672. The NLBD may have arrays with more or fewer nanocapillaries as desired. FIG. 6s illustrates a close up of a 1000 capillary array 672 of the NLBD of FIG. 6r. FIG. 6t is a photograph illustrating a NLBD 670 mounted on a circuit board 674.

FIG. 6u is a micrograph illustrating a single nanocapillary 2. In this embodiment, the nanocapillary 2 has a diameter of approximately 40 nm. However, nanocapillaries may be fabricated with larger or smaller diameters, such as between 1 to 40 nm, 5 to 25 nm, 50 to 250 nm, and 50-500 nm.

FIG. 6v is a side schematic cross section of a NLBD 670. The NLBD 670 includes a lower substrate 676 which includes microwells 677 in which a biological sample may be assayed. As discussed above, the cells may be lysed in a chamber 680 adjacent the nanocapillaries 2. In this embodiment, the bottom of the microscale wells may be formed in a separate substrate 676 from the growth substrate of the nanocapillary device. The capillary and nanoscale chamber may be removed from the growth substrate and then bonded to a separate substrate 676 containing larger (microscopic) chambers/microwells 677 defined in a process separate from the process in which the nanoscale capillaries and nanoscale chambers/wells were fabricated.

FIG. 6w is a schematic diagram illustrating an embodiment of a NLBD 670 that includes multiplexing of multiple nanocapillary arrays 640. In an embodiment, the individual arrays 640 may be independently controlled. In this manner, one or more arrays may be configured to analyse for the same or different biologicals as desired. That is, the NLBD 670 may be divided into separate parts (nanocapillary arrays 640) for performing parallelized functionality.

FIG. 6x is schematic diagram illustrating an embodiment using pressure driven flow through a nanoscale capillary. In this embodiment, uncharged molecules 20 may be streamed through the nanocapillary 2. In this embodiment, it is not necessary to form a potential gradient across the wrap around electrodes 8a, 8b to cause the molecules to flow through the nanocapillary 2. Optionally, if the molecules 20 are charged, the potentials of the wrap around electrodes 8a, 8b may be configured to aid in streaming the charged molecules 20 through the nanocapillary 2.

For the pressure driven embodiment, the transit time τ through the nanocapillary 2 is a function of the length L of the nanocapillary 2, the viscosity μ of the fluid being passed through the nanocapillary 2, the pressure drop ΔP across the nanocapillary 2 and the radius r of the nanocapillary 2 as indicated in equation 1 below:

$$τ = \frac{8 {L}^{2} μ}{Δ {Pr}^{2}} \qquad Equation 1$$

If the fluid flow in the channel network supplying the nanocapillary device is relatively large and the molecules 20 pass through the nanocapillary 2 via diffusion, the diffusion time t through the nanocapillary is a function of length x of the nanocapallary 2, the viscosity μ of the fluid containing the molecule 20, and the radius a of the molecule as indicated in equation e below:

$$t = \frac{{x}^{2}}{6 πμ a} \qquad Equation 2$$

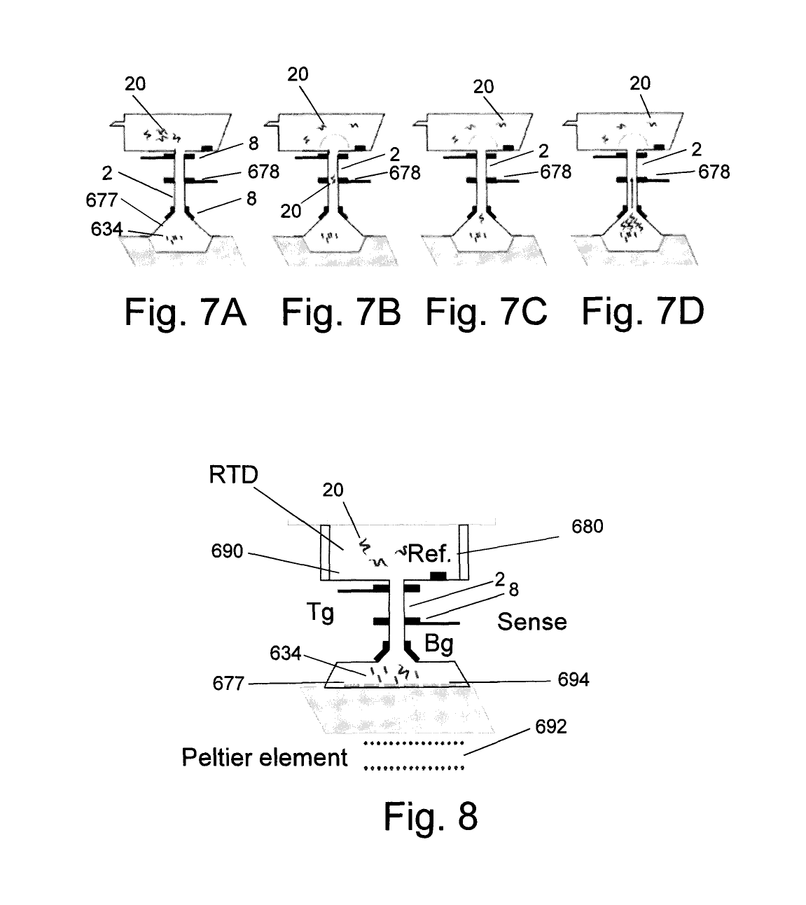

FIGS. 7A-7D are schematic diagrams illustrating an embodiment of a method of sensing charged particles moving through the nanocapillary 2 surrounded by the wrap around electrode 8 via induced charges on the wrap around electrode 8. In a first step illustrated in FIG. 7A, a DNA particle 20 is attracted into a capillary 2 using the wrap around electrodes 8. The moving DNA particle 20 passes the sensor 678 (e.g. the middle wrap around sensing electrode) and induces a charge on the wrap electrode sensor 678 over a short period of time, thereby producing a transient current response. In this embodiment, the sensor 678 senses a change in potential due to the presence of the charged DNA particle 20. As illustrated in FIG. 7C, the DNA particle 20 then passes to a microwell 677 below the nanocapillary 2 which may include primers 634. In the embodiment of the method illustrated in FIG. 7D, the DNA particles are allowed to react with the primers 634 in the microwell 677.

FIG. 8 is a schematic diagram illustrating an embodiment of a nanocapillary device connected to at least one heating element. The embodiment of this device further includes a thermal detector 690. The thermal detector 690 may be, for example, a resistive thermal detector (RTD) located in chamber 680. Example heating elements include a Peltier element 692 located below the capillary chip or an integrated (on chip) resistive heater line 694 located in (e.g. at the bottom of the) microscale chamber 677. In an alternative embodiment, the device includes a cooling element. The cooling element may be an externally mounted Peltier element 692 working with the opposite polarity as the heating element discussed above. In an alternative embodiment, the DNA particles 20 may be transferred to the nanocapillary 2 by producing a pressure difference between the top and bottom of the nanocapillary 2 rather than by changing the potentials of the wrap around electrodes 8.

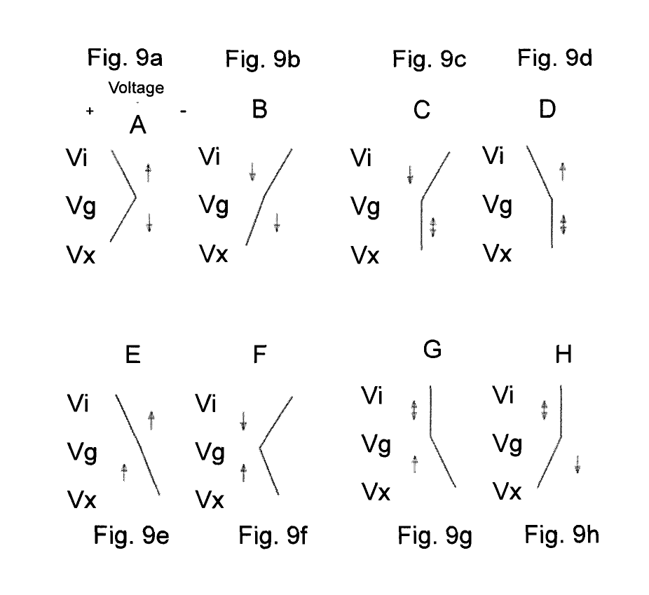

FIGS. 9a-9h are schematic diagrams illustrating the control of charged molecules in a nanocapillary device according to embodiments of the invention. As in FIGS. 2c-2f, the voltage illustrated in FIGS. 9a-9h is more positive to the left and more negative to the right. Further, in these figures, the arrows adjacent the y-axis indicate the direction of movement of the molecule under the influence of the applied potentials. Arrows pointing toward the x-axis indicate movement into the nanocapillary 20, while arrows pointing away from the x-axis indicate movement of the molecule out of the nanocapillary 20.

The embodiments illustrated in FIGS. 9a-9h may be used in the separation of positively and negatively charged molecules and size selection of molecules through charge/buffer/voltage balance or for other purposes described above. In these embodiments, both DC and AC fields may be used. The pH of the fluid may be controlled by the addition of an appropriate buffer. Further, because of the nanoscale nature of the nanocapillary device, a low capacitance tunnel junction may be formed between the upper and upper and lower wrap around electrodes 8. The low capacitance tunnel junction may be configured to form a coulomb blockade which may be used to detect charged molecules 20 as they pass through the nanocapillary 2.

In the embodiment illustrated in FIG. 9a, the respective potentials Vᵢ, V_{g}, Vₓ on the external electrode 18 and the wrap around electrodes 8a, 8b are configured to block charged molecules 20 from entering a capillary 2 open towards a single reservoir 677, 680 or exiting or emptying through capillary between two reservoirs 677, 680. In the embodiment illustrated in FIG. 9b, the potentials (e.g., Vₓ>0 Vᵢ<0, V_{g}=0 or Vₓ>V_{g}>Vᵢ) on the external electrode 18 and the wrap around electrodes 8a, 8b are configured for trapping or loading a capillary open towards a single reservoir 677, 680 or transferring a molecule 20 through a capillary 2 between two reservoirs 680 to 677. In the embodiment illustrated in FIG. 9c, the potentials (e.g., Vᵢ<0, V_{g}=Vₓ>Vᵢ e.g., V_{g}=0=Vₓ) on the external electrode 18 and the wrap around electrodes 8a, 8b are configured for retaining/blocking or loading/trapping a molecule in a capillary 2. In the embodiment illustrated in FIG. 9d, the potentials (e.g., Vᵢ>0, V_{g}=Vₓ<Vᵢ e.g., V_{g}=0=Vₓ) on the external electrode 18 and the wrap around electrodes 8a, 8b are configured to block molecules 20 from entering into a capillary 2 open towards a single reservoir 677, 680 and from entering from one side into a capillary 2 between two reservoirs 677, 680. In the embodiment illustrated in FIG. 9e, the potentials (e.g., Vᵢ>0, Vₓ<0, Vᵢ>V_{g}>Vₓ e.g., V_{g}=0) on the external electrode 18 and the wrap around electrodes 8a, 8b are configured for releasing a molecule 20 from a capillary 2 open towards a single reservoir 677, 680 or transferring the molecule through a capillary 2 between two reservoirs 677, 680. In the embodiment illustrated in FIG. 9f, the potentials (e.g., Vᵢ,Vₓ<0, V_{g}>Vᵢ,Vₓ e.g., V_{g}=0) on the external electrode 18 and the wrap around electrodes 8a, 8b are configured for positioning one or more molecules 20 in proximity to a wrap around electrode 8 of a capillary open towards a single reservoir 677, 680 or trapping/loading molecules into a capillary 20 between two reservoirs 677, 680. In the embodiment illustrated in FIG. 9g, the potentials (e.g.,Vₓ<0, Vₓ<V_{g}=Vᵢ e.g., V_{g}=Vᵢ=0) on the external electrode 18 and the wrap around electrodes 8a, 8b are configured for retaining/blocking or loading/trapping a molecule 20 in a capillary 2. In the embodiment illustrated in FIG. 9h, the potentials (e.g.,Vₓ>0, Vₓ>V_{g}=Vᵢ e.g., V_{g}=Vᵢ=0) on the external electrode 18 and the wrap around electrodes 8a, 8b are configured to block molecules 20 from entering into a capillary 2 open towards a single reservoir 677, 680 and from entering from one side into a capillary between two reservoirs 677, 680.

It is to be understood from the above that combining of different features of the nanosyringe with the nanocapillary, such as provision of an electronic probe, presence and number of protected and unprotected, as well as potential filling of the capillary with transparent, gates, metallic or semiconductor material is encompassed by the spirit of the invention. In the same context, different materials of the substrate (conventional silicon, another semiconductor or e.g. transparent material, such as glass) and different substrate functionalities, e.g. sensing or conducting properties, both inherent and added, may be readily integrated in solutions comprising nanocapillary-based nanosyringes provided with various features according to the above.

In the drawings and specification, there have been disclosed typical preferred embodiments of the invention and, although specific terms are employed, they are used in a generic and descriptive sense only and not for purposes of limitation, the scope of the invention being set forth in the following claims.

1. A device comprising at least one nanoscale capillary and means for applying an electric voltage, said means being adapted to create an electric field at least in said capillary when said electric voltage is applied, so that, when said electric voltage is applied, a charged molecule or particle placed within the created electric field can be electrically controlled.

2. A device of claim 1, wherein said charged molecule or particle is positioned in solution and said electric control of the charged molecule or particle comprises at least one of its trapping, retaining, releasing and displacement within the capillary.

3. A device of claim 2, wherein any of the trapping, retaining, releasing and displacement is determined by a level of the applied electric voltage.

4. A device of any of the claims 2-3, wherein any of the trapping, retaining, releasing and displacement is determined by a frequency of the applied electric voltage.

5. A device of any of the preceding claims, wherein said means for applying an electric voltage comprises at least two electrodes.

6. A device of any of claims 2-5, wherein said trapping can be tuned to only trap a predetermined amount of charge.

7. A fluidic network structure comprising at least one nanoscale capillary, said capillary being positioned on a fluidic channel network, the network being positioned on a substrate.

8. A fluidic network structure of claim 7, wherein the network extends in a substantially horizontal direction and the capillary extends in a substantially vertical direction.

9. A fluidic network structure of claim 7 or 8, wherein the substrate comprises electric and/or fluidic vias and/or electric circuitry.

10. A fluidic network structure of any of claims 7-9, wherein the fluidic network structure is an integral unit.

11. A fluidic network structure of claims 10, wherein said unit is made of predominantly one material.

12. A fluidic network structure of claim 11, wherein said material is a dielectric, such as Al₂O₃.

13. A fluidic network structure of any of claims 7-12, wherein the fluidic network structure is transferable from the substrate.

14. A method of manufacturing a fluidic network structure comprising at least one nanoscale capillary on a fluidic channel network, said method comprising following steps:
providing a substrate,
growing at least one vertical, essentially one-dimensional nanostructure on said substrate,
patterning a fluidic channel network,
depositing at least one layer of material creating thereby an enclosing integral unit delimited by the material layer and the substrate,
removing at least part of the interior of said enclosing integral unit so as to create said capillary and said fluidic channel network.

15. A method of claim 14, wherein the deposited layer is made of predominantly one material.

16. A method of claim 14 or 15 further comprising the step of providing means for applying voltage so that an electric field is formed at least in the capillary.

17. A method of any of claims 14-16 further comprising the step of incorporating electric and/or fluidic vias and/or electric circuitry in said substrate.

18. A nanosyringe comprising a hollow nanowire.

19. A biological material trap comprising a hollow nanowire.

20. The device of any of claims 18-19, wherein the hollow nanowire contains a gate electrode adjacent to the nanowire.

21. The device of any of claims 18-20, wherein the device is used for cell manipulation or DNA trapping, preparation and/or sequencing.

22. The device of any of claim 18 or 20, wherein the device is used for cell manipulation for drug screening, system biology, cell reprogramming, personalized medicine or tissue engineering.

23. A fluidic method for at least one of holding, capturing or releasing molecules in and out of a nanocapillary using an electric field.

24. The method of claim 23 further comprising performing lysing and/or polymerase chain reaction (PCR).

25. A fluidic device for at least one of holding, capturing or releasing molecules in and out of a nanocapillary using an electric field.

26. The device of claim 25, further comprising a heater adjacent the nanocapillary.

27. The device of claim 25, further comprising a counter electrode outside of the nanocapillary and at least one electrode adjacent to the nanocapillary.

28. The device of claim 1, wherein the nanoscale capillary comprises non-conducting sidewalls, the means for applying an electric voltage comprises of at least one electrically conducting wrap around electrode wherein the non-conducting sidewalls and the wrap around electrode form a non-conducting/conducting hetero-junction along the capillary axis.

29. The device of claim 28, wherein at least one electrically conducting electrode is located external to the nanoscale capillary.

30. The devices of claim 28 or 29, wherein the at least one nanocapillary is seamlessly connected to at least one nanoscale chamber.

31. The device of any of claims 28 to 30, where the at least one nanoscale capillary and/or at least one nanoscale chamber is seamlessly connected to a nano-channel network.

32. The device of any of claims 28 to 31, wherein applying an electrical potential difference(s) to said at least one electrically conducting wrap around electrode causes at least one charged particle to be electrically manipulated to enter, exit, or be blocked from entering or exiting the capillary.

33. The device of any of claims 28 to 32, wherein the device further comprises a second electrically conducting wrap around electrode that is configured to sense charged particles moving through the second electrically conducting wrap around electrode via induced charges on the second electrically conducting wrap around electrode.

34. The device of any of claims 28 to 33, wherein the device further comprises a second electrically conducting wrap around electrode configured as a potential probe to sense a change in the concentration of charged particles due to a chemical reaction in the nanocapillary or in an immediate vicinity of the nanocapillary.

35. The device of any of claims 28 to 34, wherein the device is connected to a micro-scale chamber and/or channel network.

36. The device of any of claims 28 to 35, wherein the device is attached to a substrate, wherein the substrate comprises an inorganic or an organic material selected from insulator, metal or semiconductor, and wherein the substrate is oblique, transparent, and/or flexible.

37. The device of claim 35, wherein the device is connected to at least one thermal detector.

38. The device of claim 35, wherein the device is connected to at least one heating element.

39. The device of claim 35, wherein the device is connected to at least one cooling element.

40. The device of claim 1, in which transfer of matter within the device is caused by pressure.

41. The device of claim 1, wherein the means for applying an electric voltage is configured to create an electric potential between the capillary and an external location, such that potential field lines originate inside the capillary and end outside the capillary.

42. The device of claim 41, further comprising means for applying an internal potential which is situated such that the potential field lines originate and end inside the capillary.

43. The device of any of claim 41 or 42, wherein at least one of the means for applying an electric voltage or the means for applying an internal potential is an electrode.

44. The device of claim 43, wherein at least one of the means for applying an electric voltage or the means for applying an internal potential is a wrap around electrode.

45. The device of claim 44, wherein the at least one nanoscale capillary has non-conducting sidewalls and at least one electrically conducting wrap around electrode forms a non-conducting/conducting hetero-junction along the capillary axis.

46. The device of any of claims 41 to 44, wherein a charged molecule or particle is electrically controlled to exit, enter, be trapped, be retained, be released, be blocked from entering or be moved within the capillary.

47. The device of any of claims 41 to 46, wherein the at least one capillary is open in both ends.

48. The device of claim 47, wherein the device is configured to transfer fluid through a pressure driven flow.

49. The device of any of claims 41 to 48, further comprising an additional means for applying electric potential configured such that the field lines may end externally to the capillary at opposite ends of the capillary.

50. The device of any of claims 41 to 49, wherein the capillary forms a syringe for injection and extraction of molecules into biological matter.

51. The device of any of claims 41 to 50, further comprising an electrical sensor configured to detect charges released or charges passing the electrode in the capillary.

52. The device of claim 51, wherein the sensor comprises a wrap around electrode around the capillary.

53. An array structure comprising the device of any of claims 28 to 52.

54. A fluidic system comprising the array structure of claim 53, and further comprising at least one adjacent structure comprising any of a chamber, a sample plate, a biocell holder, a channel network, temperature sensor, heating element, cooling element or an optical detector.

55. The fluidic system of claim 54, wherein either the array structure or the at least one adjacent structure is divided into separate parts for parallelized functionality.

56. The fluidic system of any of claim 54 or 55, wherein the at least one adjacent structure and the array structure are detachable from each other.

57. The fluidic system of any of claims 54 to 56, wherein the device of the array structure is seamlessly connected to one of the at least one adjacent structures, the adjacent structure being one of a chamber or a channel network.

58. The fluidic system of any of claims 54 to 57, wherein the system is connected to a substrate, wherein the substrate comprises an inorganic or an organic material selected from insulator, metal, or semiconductor, and wherein the substrate is oblique, transparent and/or flexible.

59. The fluidic system of any of claims 54 to 58, wherein the array structure is located in a membrane.

60. The device of claim 28, wherein the device further comprises a second electrically conducting wrap around electrode, the second electrically conducting wrap around electrode spaced apart from the at least one electrically conducting wrap around electrode.

61. The biological material trap of claim 19, wherein the biological material trap comprises a DNA trap.

62. A fluidic method for at least one of holding, capturing or releasing molecules in and out of a nanocapillary using the device of any of claim 1-6 or 28-61.
